(19) United States
(12) Patent Application Publication
(10) Pub. No.: US 2008/0051900 A1
(43) Pub. Date: Feb. 28, 2008

(54) Spinal Prosthesis with Offset Anchors

(75) Inventors: Malan de Villiers, Irene (ZA); David Hovda, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpinalMotion, Inc., Mountain View, CA (US)

(21) Appl. No.: 11/829,054

(22) Filed: Jul. 26, 2007

Related U.S. Application Data
(60) Provisional application No. 60/820,769, filed on Jul. 28, 2006.

(51) Int. Cl. A61F 2/44; A61B 17/56

(52) U.S. Cl. 623/17.16; 606/72

(57) ABSTRACT

An implanted intervertebral prosthesis includes first and second components adapted to attach to a first vertebra and a second vertebra, respectively, that define an intervertebral space. The prosthesis includes a first anchor structure on the first component adapted to enter a grove formed in the first vertebra, and a second anchor structure on the second component adapted to enter a grove formed in the second vertebra. The first anchor structure is offset from the second anchor structure to provide separation of the grooves to preserve vertebral thickness and avoid vertebral splitting. The offset anchor structures can be symmetrically disposed about a midline of the prosthesis or asymmetrically disposed about the midline. In some embodiments, the offset anchor structures comprise elongate anchors shaped as fins or keels. In other embodiments, the offset anchor structures comprise rows of pillars disposed in rows.

### CROSS REFERENCE TO RELATED APPLICATIONS

The present non-provisional application claims the benefit under 35 USC 119e of U.S. Appl. No. 60/820,769 (attorney docket no. 022031-002000US), entitled “Spinal Prosthesis with Offset Anchors,” filed on Jul. 28, 2006, the full disclosure of which is incorporated herein by reference.

### BACKGROUND OF THE INVENTION

1. Field of the Invention

This invention relates to medical devices and methods. More specifically, the invention relates to restoration of spinal motion with a prosthetic disc for intervertebral insertion, such as in the lumbar and cervical spine.

In the event of damage to a lumbar or cervical intervertebral disc, one possible surgical treatment is to replace the damaged disc with an intervertebral disc prosthesis. Several types of intervertebral disc prostheses are currently available. One type available under the trademark SB Charite (DePuy Spine, a division of Johnson & Johnson, New Brunswick, N.J.), includes upper and lower prosthesis plates or shells which engage the adjacent vertebral bodies with a low friction core between the plates. [See U.S. Pat. No. 4,759,566; EP 1142544A1 and EP 1250898A1] Many prosthetic discs use protruding anchors to anchor the endplates to the adjacent vertebra, for example, an elongate anchor adapted to enter a groove cut into a vertebra as described in U.S. Pat. No. 4,863,477. While elongate fins, keels and other anchors have generally been successful in anchoring endplates to vertebra, clinical trials with large numbers of patients have shown that in rare cases complications can arise. In particular, the elongate anchors prosthetic discs are sometimes placed in adjacent intervertebral spaces on opposite ends of a vertebra, which is referred to as “stacking”. The vertebra positioned between the adjacent prosthetic disks can split. Work in relation with the present invention suggests that this splitting of a vertebra with stacked prosthesis may be caused by grooves cut in the same plane on the upper and lower surfaces of vertebra so that the thickness of the vertebral body is decreased. Also, placing the grooves in the same plane on upper and lower surfaces of a vertebra may result in the formation of a fracture plane. This complication is clearly undesirable and typically requires surgical intervention. Another rare complication that can arise is that the endplates of the implanted prosthetic disc can slip, causing patient discomfort and requiring surgical intervention.

Another prosthetic approach has been to fuse the vertebrae, for example with transforaminal lumbar interbody fusion (TLIF) surgery or posterior lumbar interbody fusion (PLIF) surgery. Fusion surgery generally requires at least partial removal of one or more facet joints, bone grafting, and support with a fusion cage to stop the motion at that segment. Although the fusion cages can be inserted from the back of the patient, such prostheses generally do not provide a flexible joint at the damaged disc site or other implant site. Thus a potential disadvantage of these fusion approaches is that spinal motion is not restored at the intervertebral joint.

In light of the above, it would be desirable to provide improved prostheses, particularly surgical prostheses which at least partially restore motion and provide consistent attachment of the prosthetic endplates to vertebrae without compromising strength of the vertebrae.

2. Description of the Background Art

Published U.S. patent applications published under Patent Publication Nos. 2002/0035400A1 and 2002/0128715A1 describe disc implants which comprise opposing plates with a core between them over which the plates can slide. Expandable intervertebral prostheses are described in U.S. Appl. No. 60/744710 (attorney docket no. 022031-001900US), entitled “Spinal Disc Arthroscopy,” filed on Apr. 12, 2006; and U.S. Appl. No. 60/746731 (attorney docket no. 022031-001910US), entitled “Spinal Disk Arthroscopy,” filed on May 8, 2006, the full disclosures of which are incorporated herein by reference. Other patents related to intervertebral disc prostheses include U.S. Pat. Nos. 4,759,766; 4,863,477; 4,997,432; 5,035,716; 5,071,437; 5,258,031; 5,370,697; 5,401,269; 5,507,816; 5,534,030; 5,556,431; 5,674,296; 5,676,701; 5,676,702; 5,702,450; 5,797,909; 5,824,094; 5,865,846; 5,989,291; 6,001,130; 6,022,376; 6,039,763; 6,096,038; 6,139,579; 6,156,067; 6,162,252; 6,315,797; 6,348,071; 6,368,350; 6,416,551; 6,592,624; 6,607,558; 6,706,068; 6,740,118; and 6,936,071. Other U.S. patent applications related to intervertebral disc prostheses include Patent Publication Nos.: 2001/0016773; 2002/0035400; 2002/0128715; 2003/0009224; 2003/0074076; 2003/0100951; 2003/0135277; 2003/0191536; 2003/0208271; 2003/0199982; 2004/0030391; 2004/0073312; 2004/0143270; 2004/0176843; 2005/0043800; 2005/0085917; 2005/0107881; 2005/0149189; 2005/0192586; 2005/0261772; and 2006/0041313. Other related patents and applications include WO 01/01893A1, WO 2005/053580, EP 1344507, EP 1344506, EP 1250898, EP 1306064, EP 1344508, EP 1344493, EP 1417940, EP 1142544, and EP 0333990.

### BRIEF SUMMARY OF THE INVENTION

Embodiments of the present invention provide an implanted intervertebral prosthesis which restores motion, provide improved attachment of the prosthesis to the adjacent vertebrae, and may decrease the possibility of vertebral splitting where multiple adjacent implants are used. The prosthesis includes first and second components adapted to attach to a first vertebra and a second vertebra, respectively, that define the intervertebral space. The prosthesis includes at least a first anchor structure on the first component. The first anchor structure is adapted to enter a grove formed in the first vertebra. The prosthesis has a midline, and the midline of the prosthesis extends proximally and distally along the prosthesis and often corresponds to a midline of the vertebrae. The anchor structure can be asymmetrically disposed about the midline of the prosthesis so that a grove cut in an end of a vertebra is asymmetric about the midline of the vertebra. The prosthesis can also include more than one anchor structure on one of the components so that the groves cut in an end of the vertebra are symmetrically disposed on opposite sides of the midline of the vertebra.

By “asymmetric” or “asymmetrically” it is meant that the anchor structure will be laterally offset from the prosthesis midline (and thus requiring cutting of a groove in the vertebra for placement) and that there will be no second anchor structure having the same or similar dimensions, located symmetrically on the same component. Usually, but not necessarily, there will be no anchor structures which require a precut grove on the vertebra for placement located on the opposite side of the midline on the same component. In contrast, there will frequently be a second anchor structure located on the opposite side of the center line of the other component, very often being symmetrically placed with respect to the first anchor structure.

In one aspect, embodiments of the present invention comprise an intervertebral prosthesis with an offset anchor structure that asymmetrically anchors the prosthesis. The intervertebral prosthesis comprises an upper component that is adapted to engage an upper vertebra, and a lower component that is adapted to engage a lower vertebra. The first and second components are adapted to engage each other or an intermediate member to form an articulate joint between the vertebrae. An asymmetric anchor structure is disposed on one of the components. The asymmetric anchor structure is adapted to enter an asymmetric groove formed in the upper vertebra or the lower vertebra. The prosthesis comprises a midline and the asymmetric anchor structure is disposed on a line parallel to but laterally offset from the midline.

In specific embodiments the asymmetric anchor structure has a height and a width, and the height is greater than twice the width. The asymmetric anchor structure can comprise a continuous structure that extends in proximal and distal directions along one of the components, for example a fin or keel that extends in proximal and distal directions along one of the components. The asymmetric anchor structure can comprise a discontinuous structure that extends in proximal and distal directions along one of the components, for example a plurality of anchors disposed along the line parallel to but offset from the midline. In some embodiments, the offset anchor structures comprise continuous elongate anchors shaped as fins or keels. In other embodiments, the offset anchor structures comprise discontinuous anchor structures, for example rows of pillars disposed along the line parallel to the midline.

In other embodiments an intervertebral prosthesis comprises a first asymmetric anchor structure and a second asymmetric anchor structure. An upper component is adapted to engage an upper vertebra. A lower component is adapted to engage a lower vertebra. The upper component and the lower component are adapted to engage each other or an intermediate member to form an articulate joint. The first asymmetric anchor structure is disposed on the upper component. The first asymmetric anchor structure is adapted to enter an asymmetric groove formed in the upper vertebra. The prosthesis comprises a midline and the anchor structure is disposed on a first line parallel to but laterally offset from the midline. A second asymmetric anchor structure is disposed on the lower component. The second asymmetric anchor structure is adapted to enter an asymmetric groove formed in the lower vertebra. The second asymmetric anchor structure is disposed along a second line on a side of the midline opposite to that of the first asymmetric anchor structure.

In specific embodiments the second line is parallel to the first line. The first and second anchor structures are laterally offset from the midline by equal distances. The first and second anchor structures each has a height and a width, and the height is greater than twice the width. The first and second anchor structures can comprise continuous structures that extend in proximal and distal directions along the components, for example fins that extend in proximal and distal directions along each component. The first and second anchor structures can also comprise discontinuous structures that extend in proximal and distal directions along the components, for example a plurality of anchors disposed along each of the first and second lines.

In other embodiments an intervertebral prosthesis comprises at least one anchor that is disposed on a midline of the prosthesis and at least one pair of anchors offset from the at least one anchor disposed on the midline. A first component has the at least one anchor disposed thereon. The at least one anchor is adapted to enter a groove in a first vertebra. A second component has the at least one pair of anchors disposed thereon. The at least one pair of anchors is adapted to enter a pair of grooves in a second vertebra. The first component and the second component are adapted to engage each other or an intermediate member to form an articulate joint. The at least one anchor of the first component is disposed on the midline, and each anchor of the at least one pair is disposed on opposite sides of the midline.

In specific embodiments, the at least one anchor of the first component is offset from each anchor of the at least one pair of anchors. Each anchor of the at least one pair is shorter in height than the at least one anchor disposed on the midline. Each anchor of the at least one pair is separated from the midline by a distance, and each anchor of the at least one pair is shorter than the anchor on the midline by an amount in proportion to the distance. The proportional amount is from 5 to 50% of the distance, and can be from 10% to 40% of the distance. Each anchor of the at least one pair is offset from the midline by a similar distance, and each anchor of the at least one pair has a similar height. The at least one anchor is disposed on the midline can comprises several anchors disposed on the midline and adapted to enter the groove in the first vertebra. The at least one pair of anchors can comprise several pairs of anchors adapted to enter the pair of grooves formed in the second vertebra.

In another aspect, embodiments of the present invention comprise a method for anchoring an intervertebral prosthesis within an intervertebral space between a pair of vertebral bodies. A groove is cut in a first end of at least one of the vertebral bodies. The at least one vertebral body has a second opposing end with a groove which has been previously formed thereon to receive an other intervertebral prosthesis. The prosthesis is introduced into the intervertebral space between the vertebral bodies so that at least one anchor structure enters the groove in the first end. The groove cut in the first end is offset from the groove cut in the second end.

In specific embodiments, the grooves cut in the first and second ends are offset to preserve thickness of the vertebral body. The grooves cut in the first and second ends are offset to avoid cutting the vertebral body along the midlines. The prosthesis and the other prosthesis are disposed in adjacent intervertebral spaces separated by the at least one vertebral body with the grooves cut in the first and second ends. The first groove and second groove can be cut during the same surgical procedure. Also, the first groove and second groove can be cut during separate surgical procedures. The groove cut in the first end is cut along a first plane and the groove cut in the second end is along a second plane, and the first plane is offset from the second plane. The anchor structure can be discontinuous and several anchors enter the groove in the first end as the prosthesis is introduced into the intervertebral space.

In other embodiments include a method for placing intervertebral prostheses between vertically adjacent vertebrae. Laterally staggered grooves are cut in upper and lower vertebral surfaces of at least two vertically adjacent vertebral spaces. No grove is cut on any surface on an opposite side of centerline. At least first and second prostheses are introduced into the vertically adjacent spaces. An asymmetric anchor structure on each of the first and second prostheses enters a grove cut into the vertebral surfaces.

### BRIEF DESCRIPTION OF THE DRAWINGS

FIG. 1A shows an intervertebral prosthesis with symmetric offset anchor structures implanted between adjacent vertebrae according to an embodiment of the present invention;

FIG. 1B shows an isometric view of the intervertebral prosthesis as in FIG. 1A according to an embodiment of the present invention;

FIG. 1C shows an end view of the intervertebral prosthesis as in FIGS. 1A and 1B according to an embodiment of the present invention;

FIG. 1D shows a side view of the intervertebral prosthesis as in FIGS. 1A to 1C according to an embodiment of the present invention;

FIG. 2A shows an intervertebral prosthesis with asymmetric anchor structures implanted between adjacent vertebrae according to an embodiment of the present invention;

FIG. 2B shows an isometric view of the intervertebral prosthesis as in FIG. 2A according to an embodiment of the present invention;

FIG. 2C shows an end view of the intervertebral prosthesis as in FIGS. 2A and 2B according to an embodiment of the present invention;

FIG. 2D shows a side view of the intervertebral prosthesis as in FIGS. 2A to 2C according to an embodiment of the present invention;

FIG. 3 shows an intervertebral prosthesis with symmetric offset anchor structures comprising several pillars according to an embodiment of the present invention; and

FIG. 4 shows an intervertebral prosthesis with asymmetric anchor structures comprising several pillars according to an embodiment of the present invention.

### DETAILED DESCRIPTION OF THE INVENTION

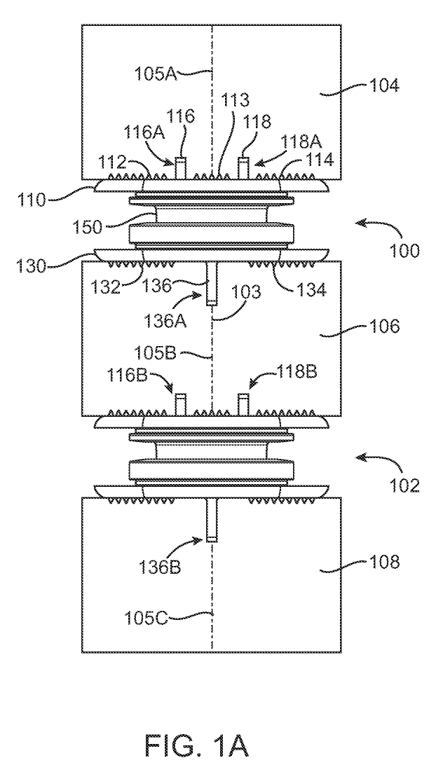

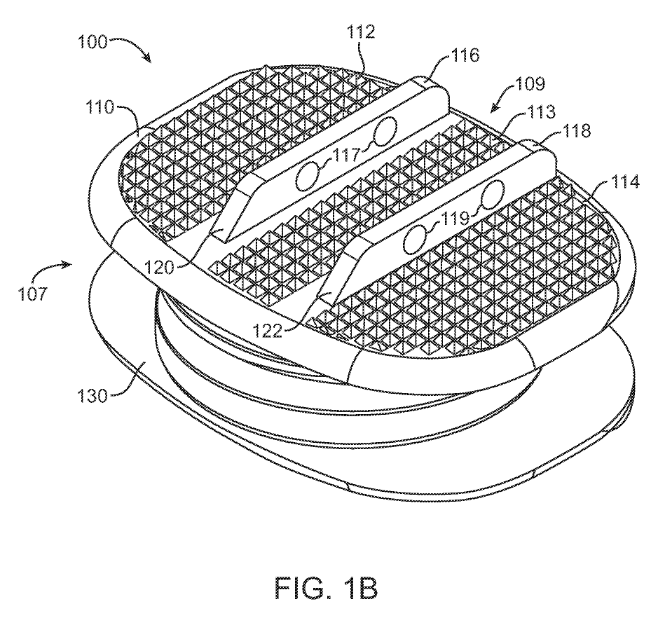

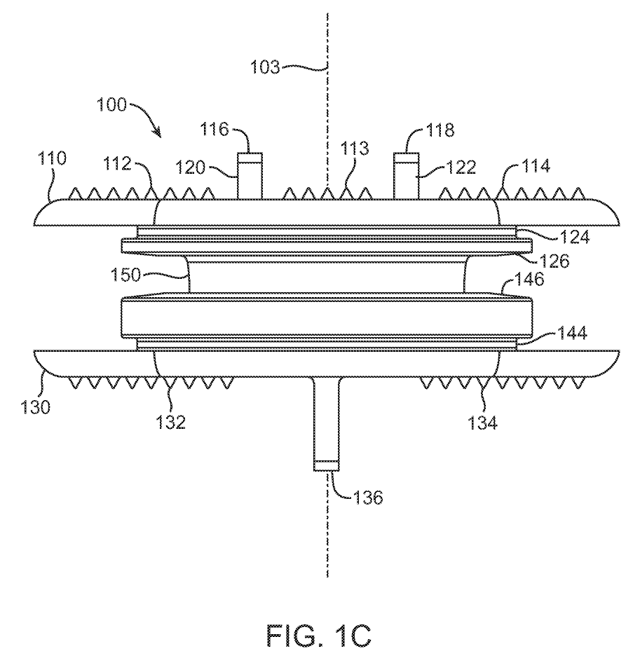

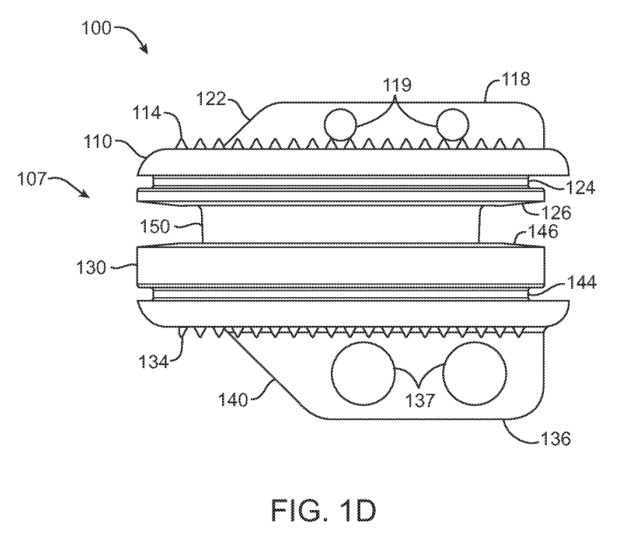

FIG. 1A shows an intervertebral prosthesis 100 with symmetric offset continuous anchor structures implanted between adjacent vertebrae according to an embodiment of the present invention. An upper vertebra 104, a middle vertebra 106 and a lower vertebra 108 are components of a patient spine and include midlines 105A, 105B and 105C respectively. Upper vertebra 104 and middle vertebra 106 define an intervertebral space where intervertebral prosthesis 100 is located. Intervertebral prosthesis 100 includes a midline 103 that coincides with a midline 105B of middle vertebra 106. Intervertebral prostheses 100 includes an upper component 110 that engages upper vertebra 104 and a lower component 130 that engages middle vertebra 106. Intervertebral prosthesis 100 includes an intermediate member 150, or mobile core, disposed between upper component 110 and lower component 130. Upper component 110, intermediate member 150 and lower component 130 form articulate joint so that relative motion of the vertebrae is restored following implantation of the intervertebral prosthesis, for example as described in U.S. Pat. No. 4,759,766; U.S. application Ser. No. 10/855,817, entitled “Prosthetic Disc for Intervertebral Insertion,” filed May 26, 2004, U.S. Pub. No. 2005/0021146 (attorney docket no. 022031-000210US); U.S. application Ser. No. 10/855,253, entitled “Prosthetic Disc for Intervertebral Insertion,” filed May 26, 2004, U.S. Pub. No. 2005/0021145 (attorney docket no. 022031-000310US); and U.S. application Ser. No. 10/903,913, entitled “Intervertebral Prosthetic Disc with Metallic Core,” filed Jul. 30, 2004, U.S. Pub. No. 2006/0025862 (attorney docket no. 022031-001400US); the full disclosures of which are incorporated herein by reference. Upper component 110 includes a lower bearing surface that engages an upper surface of intermediate member 150. Lower component 130 includes an upper bearing surface that engages a lower bearing surface of intermediate member 150. In alternate embodiments the upper component and lower component bear directly against one another without an intermediate member, for example as described in U.S. Pat. Nos. 5,258,031; 5,314,477; 5,676,701; and 6,936,071. Another intervertebral prosthesis 102 is located in an intervertebral space defined by middle vertebra 106 and lower vertebra 108.

Intervertebral prosthesis 100 includes structures disposed on upper component 110 and lower component 130 to anchor the prosthesis in the intervertebral space. A pair of elongate anchors includes upper elongate anchor 116 and upper elongate anchor 118. Upper elongate anchors 116 and 118 are continuous anchors structures. These anchors are formed as elongate fins, or keels, and are adapted to enter grooves formed in a patient vertebra, for example as described in U.S. Pat. Nos. 5,314,477; 6,740,118; and 6,936,071. Several peripheral serrations are provided to anchor intervertebral prosthesis 100 in the intervertebral space. Peripheral serrations 112 are disposed peripherally to elongate anchor 116 and each serration includes a square base that extends to a point so that each serration is shaped like a pyramid with a height approximately the same as a width across the base. Central serrations 113 are disposed between elongate anchor 116 and elongate anchor 118 and each serration is shaped similarly to peripheral serrations 112. Peripheral serrations 114 are disposed peripherally to elongate anchor 118 and are shaped similarly to peripheral serrations 112. Peripheral serrations 112 and 114 and central serrations 113 each include several rows of serrations disposed parallel to elongate anchors 116 and 118. Lower component 130 includes an elongate anchor 136. Elongate anchor 136 is a continuous anchor structure. Elongate anchor 136 is centered on midline 103 so that at least a portion of elongate anchor 136 overlaps with midline 103. Elongate anchor 136 is formed as an elongate fin, or keel and adapted to enter a groove formed in a patient vertebra. Lower component 130 includes serrations 132 and serrations 134. Serrations 132 are disposed on an opposite side of component 130 from serrations 134. Serrations 132 and 134 each includes several rows disposed parallel to elongate anchor 136 similar to the serrations on upper component 110.

Several grooves are formed in upper vertebra 104, middle vertebra 106 and lower vertebra 108 to anchor prosthesis 100 and prosthesis 102. A pair of grooves formed in upper vertebra 104 includes groove 116A and groove 118A. Grooves 116A and 118A are each disposed on opposite sides of midline 105A and separated from the midline by the same distance. Grooves 116A and 118A are formed to receive elongate anchors 116 and 118, respectively. Middle vertebra 106 has a groove 136A formed thereon. Groove 136A is disposed on midline 105B and formed to receive elongate anchor 136. Each of grooves 116A, 118A and 136A are parallel as are elongate anchors 116, 118 and 136, respectively. A pair of grooves formed in middle vertebra 106 includes groove 116B and groove 118B. Grooves 116B and 118B are each disposed on opposite sides of midline 105B and separated from the midline by the same distance. Grooves 116B and 118B are formed to receive elongate anchors similar to elongate anchors 116 and 118, respectively. Lower vertebra 108 has a groove 136B formed thereon. Groove 136B is disposed on midline 105C and formed to receive an elongate anchor similar to elongate anchor 136. Each of grooves 116A, 118A and 136A are parallel as are elongate anchors 116, 118 and 136, respectively.

The elongate anchor structures of the intervertebral prostheses and the grooves formed in the vertebrae that receive these structures are offset to avoid vertebral splitting, for example by preserving thickness of the vertebra between grooves. Groove 136A is located on an opposite end of vertebra 106 from grooves 116B and 118B. Groove 136A is formed in an upper surface of vertebra 106 and located on midline 105B. Grooves 116B and 118B formed in a lower surface of vertebra 106 and are offset from midline 105B. Thus, groove 136A is offset from grooves 116B and 118B. This offset of groove 136A avoids grooves 116B and 118B and preserves thickness of vertebra 106, for example as compared to upper and lower grooves both formed on midline 105B. This preservation of thickness of vertebra 106 helps to avoid, or at least decrease, the chances of vertebral splitting.

FIG. 1B shows an isometric view of intervertebral prosthesis 100 as shown in FIG. 1A according to an embodiment of the present invention. Prosthesis 100 includes a distal end 107 that leads as the prosthesis is inserted into the intervertebral space and a proximal end 109 that lags as prosthesis 100 is inserted into the intervertebral space. Elongate anchor 116 includes a chamfer 120. Chamfer 120 and chamfer 122 facilitate insertion of elongate anchor 116 and 118, respectively, as prosthesis 100 is inserted into the intervertebral space. Elongate anchor 116 includes holes 117 formed thereon, and elongate anchor 118 includes holes 119 formed thereon. Holes 117 and 119 permit bone to grow through the anchor that may provide improved anchoring of the bone to the implant. Peripheral serrations 112 and 114 and central serrations 113 each include several parallel rows of serrations in which the rows are parallel to elongate anchors 116 and 118

FIG. 1C shows an end view of intervertebral prosthesis 100 as in FIGS. 1A and 1B according to an embodiment of the present invention. Upper component 110 includes a flange 126 and a groove 124 formed thereon. Lower component 130 includes a groove 144. Groove 124 and groove 144 are adapted to receive an instrument that is used to insert intervertebral prosthesis 100 into the intervertebral space, for example as described in U.S. application Ser. No. 10/913,780, entitled “Methods and Apparatus for Intervertebral Disc Prosthesis Insertion”, filed Aug. 6, 2004 (attorney docket no., 022031-001000US); U.S. application Ser. No. 11/187,733, entitled “Intervertebral Prosthesis Placement Instrument,” filed Jul. 21, 2005 (attorney docket no. 022031-001100US), the full disclosures of which are incorporated herein by reference. Lower component 130 includes a peripheral retaining ring 146 that retains intermediate member 150. In alternate embodiments, other retentions structures are used to retain the mobile core.

FIG. 1D shows a side view of intervertebral prosthesis 100 as in FIGS. 1A to 1C according to an embodiment of the present invention. Lower component 130 includes elongate anchor 136 with holes 137 formed thereon. Holes 137 permit bone to grow into the elongate anchor and can improve attachment of the lower component to vertebra 106. Elongate anchor 136 includes a chamfer 140 formed thereon to facilitate insertion of elongate anchor 136 into groove 136A.

The symmetric anchor structures of prosthesis 100 shown in FIGS. 1A to 1D are symmetrically arranged about midline 103. Midline 203 is a plane that extends vertically through the implant in proximal and distal directions. Anchors that are symmetrically arranged about the midline are mirror images of each other about the midline. An anchor that is centered on the midline has a first side and a second side, and the first side of the anchor is a mirror image of the second side of the anchor. Thus, a single anchor disposed on the midline is a symmetrical anchor structure, for example anchor 136. With symmetric anchor structures, each anchor structure that is displaced from the midline is mirror image of another anchor structure, for example anchors 116 and 118. Thus, if one were to fold the page about the midline and place the folded page together, each anchor structure would overlap with another anchor structure. For example, elongate anchor 116 is located on a first side of midline 103, and elongate anchor 118 is located on a second side of midline 103. The distance from midline 103 to elongate anchor 116 is the same as the distance from midline 103 to offset anchor 118 so that elongate anchor 116 and elongate anchor 118 are symmetrically disposed about midline 103. Similarly, peripheral serrations 112 are disposed on the first side of midline 103 and peripheral serrations 114 are disposed on the second side of midline 103 so that peripheral serrations 112 and peripheral serrations 114 are symmetrically disposed about midline 103.

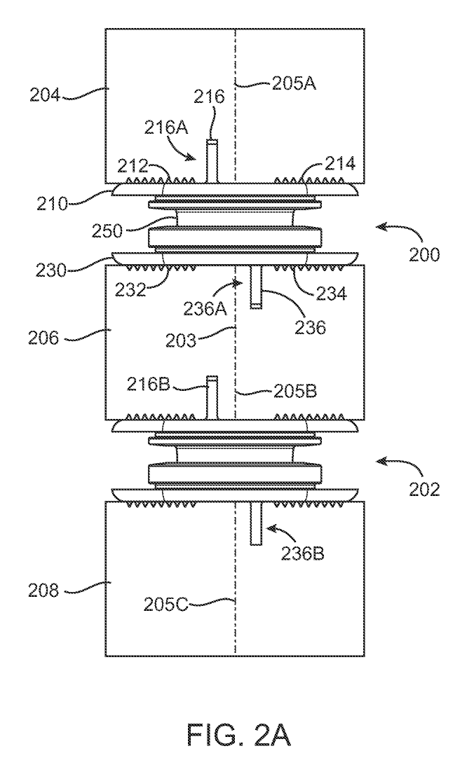

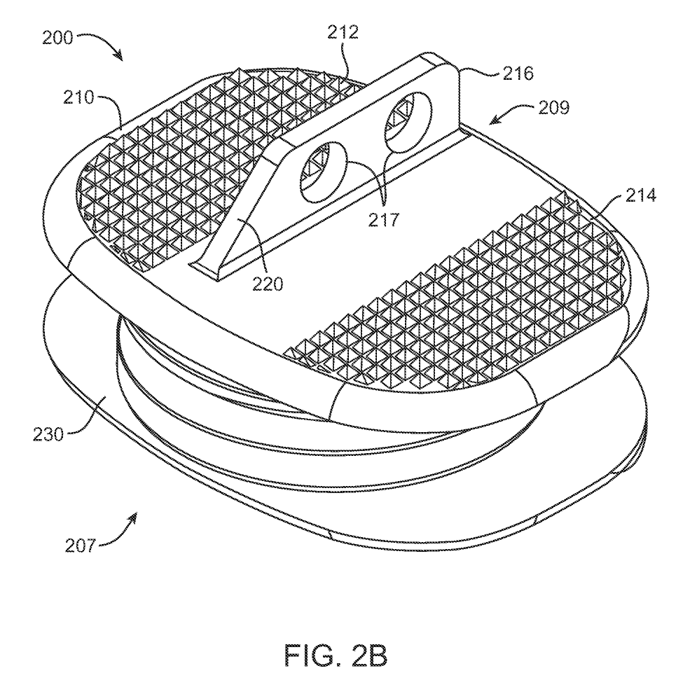

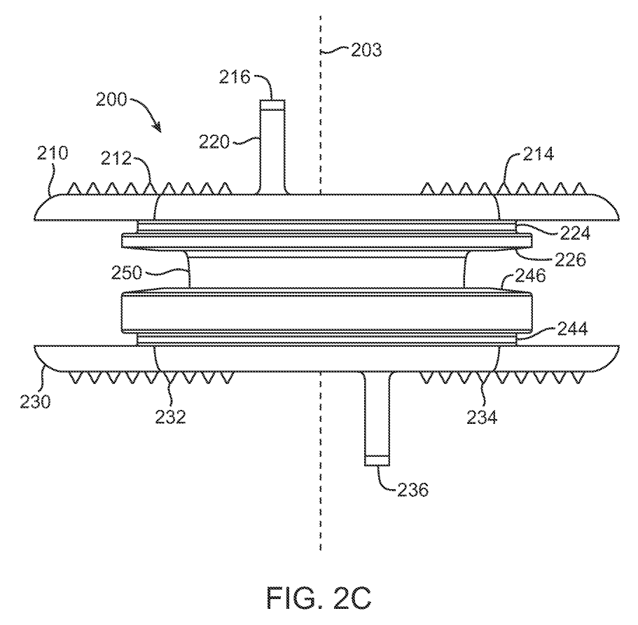

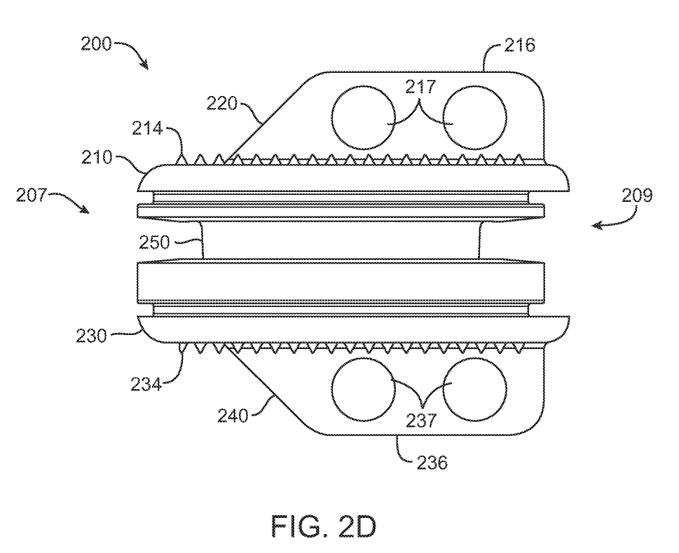

FIG. 2A shows an intervertebral prosthesis 200 with asymmetric continuous anchor structures implanted between adjacent vertebrae according to an embodiment of the present invention. An upper vertebra 204, a middle vertebra 206 and a lower vertebra 208 are components of a patient spine and include midlines 205A, 205B and 205C respectively. Upper vertebra 204 and middle vertebra 206 define an intervertebral space where intervertebral prosthesis 200 is located. Intervertebral prosthesis 200 includes a midline 203 that coincides with a midline 205B of middle vertebra 206. Intervertebral prostheses 200 includes an upper component 210 that engages upper vertebra 204 and a lower component 230 that engages middle vertebra 206. Intervertebral prosthesis 200 includes an intermediate member 250, or mobile core, disposed between upper component 210 and lower component 230. Upper component 210, intermediate member 250 and lower component 230 form articulate joint so that relative motion of the vertebrae is restored following implantation of the intervertebral prosthesis. Upper component 210 includes a lower bearing surface that engages an upper surface of intermediate member 250. Lower component 230 includes an upper bearing surface that engages a lower bearing surface of intermediate member 250. In alternate embodiments the upper component and lower component bear directly against one another without an intermediate member. Another intervertebral prosthesis 202 is located in an intervertebral space defined by middle vertebra 206 and lower vertebra 208.

Intervertebral prosthesis 200 includes structures disposed on upper component 210 and lower component 230 to anchor the prosthesis in the intervertebral space. Upper component 210 includes an upper elongate anchor 216. Upper elongate anchor 216 is a continuous anchor structure. This anchor is formed as an elongate fin, or keel, and is adapted to enter a groove formed in a patient vertebra. Peripheral serrations are provided to anchor intervertebral prosthesis 200 in the intervertebral space. Peripheral serrations 212 are disposed peripherally to elongate anchor 216 and each serration includes a square base that extends to a point so that each serration is shaped like a pyramid with a height approximately the same as a width across the base. Peripheral serrations 214 are disposed peripherally to elongate anchor 216 and are shaped similarly to peripheral serrations 212. Peripheral serrations 212 and 214 each include several rows of serrations disposed parallel to elongate anchor 216. Lower component 230 includes an elongate anchor 236. Elongate anchor 236 is a continuous anchor structure. Elongate anchor 236 is formed as an elongate fin, or keel and adapted to enter a groove formed in a patient vertebra. Elongate anchor 236 is offset from midline 203 and asymmetrically disposed about midline 203. Lower component 230 includes serrations 232 and serrations 234. Serrations 232 are disposed on an opposite side of component 230 from serrations 234. Serrations 232 and 234 each includes several rows disposed parallel to elongate anchor 236 similar to the serrations on upper component 210.

Several grooves are formed in upper vertebra 204, middle vertebra 206 and lower vertebra 208 to anchor prosthesis 200 and prosthesis 202. Upper vertebra 204 includes groove 216A formed thereon. Groove 216A is disposed on one side of midline 205A to asymmetrically anchor upper component 210 to upper vertebra 204. Groove 216A is formed to receive elongate anchor 216. Middle vertebra 206 has a groove 236A formed thereon. Groove 236A is disposed one side of midline 205B to asymmetrically anchor lower component 230 to middle vertebra 206. Each of grooves 216A and 236A is parallel as are elongate anchors 216 and 236, respectively. Middle vertebra 206 includes groove 216B formed thereon. Grooves 216B is disposed on one side of midline 205B to asymmetrically anchor intervertebral prosthesis 202 in the intervertebral space. Groove 216B is formed to receive an elongate anchors similar to elongate anchors 216. Lower vertebra 208 has a groove 236B formed thereon. Groove 236B is disposed on one side of midline 205C to asymmetrically anchor intervertebral prosthesis 202 in the intervertebral space. Each of grooves 216A and 236A are parallel as are elongate anchors 216 and 236. In alternate embodiments, the elongate anchors and grooves of an upper component are not parallel to the elongate anchors and grooves of the lower component.

The elongate anchor structures of the intervertebral prostheses and the grooves formed in the vertebrae that receive these structures are offset to avoid vertebral splitting, for example by preserving thickness of the vertebra between grooves. Groove 236A is located on an opposite end of vertebra 206 from grooves 216B. Groove 236A is formed in an upper surface of vertebra 206 and located offset from midline 205B. Groove 216B is formed in a lower surface of vertebra 206 and is offset from midline 205B. Thus, groove 236A is offset from groove 216B. This offset of groove 236A avoids groove 216B and preserves thickness of vertebra 206, for example as compared to upper and lower grooves both on midline 205B. This preservation of thickness of vertebra 206 helps to avoid, or at least decrease, the chances of vertebral splitting.

FIG. 2B shows an isometric view of intervertebral prosthesis 200 as shown in FIG. 2A according to an embodiment of the present invention. Prosthesis 200 includes a distal end 207 that leads as the prosthesis is inserted into the intervertebral space and a proximal end 209 that lags as prosthesis 200 is inserted into the intervertebral space. Elongate anchor 216 includes a chamfer 220. Chamfer 220 facilitates insertion of elongate anchor 216 into groove 216A as the prosthesis is advanced into the groove. Elongate anchor 216 includes holes 217 formed thereon. Holes 217 permit bone to grow through the anchor that may provide improved anchoring of the bone to the implant. Peripheral serrations 212 and 214 each include several parallel rows of serrations in which the rows are parallel to elongate anchor 216

FIG. 2C shows an end view of intervertebral prosthesis 200 as in FIGS. 2A and 2B according to an embodiment of the present invention. Upper component 210 includes a flange 226 and a groove 224 formed thereon. Lower component 230 includes a groove 244. Groove 224 and groove 244 are adapted to receive an instrument that is used to insert intervertebral prosthesis 200 into the intervertebral space. Lower component 230 includes a peripheral retaining ring 246 that retains intermediate member 250. In alternate embodiments, other retentions structures are used to retain the mobile core 17.

Each anchor of the pair of anchors that includes anchor 116 and anchor 118 is shorter in height than anchor 136 disposed on midline 103. Anchors 116 and 118 are separated from the midline by the same distance. Anchors 116 and 118 are shorter than the anchor 136 by an amount in proportion to the distance. The proportional amount is from 20 to 80% of the distance, often from 30% to 70% of the distance, for example 50% of the distance.

FIG. 2D shows a side view of intervertebral prosthesis 200 as in FIGS. 2A to 2C according to an embodiment of the present invention. Lower component 230 includes elongate anchor 236 with holes 237 formed thereon. Holes 237 permit bone to grow into the elongate anchor and can improve attachment of the lower component to vertebra 206. Elongate anchor 236 includes a chamfer 240 formed thereon to facilitate insertion of elongate anchor 236 into groove 236A.

The asymmetric anchor structures of prosthesis 200 shown in FIGS. 2A to 2D are asymmetrically arranged about midline 203. Midline 203 is a plane that extends vertically through the implant in proximal and distal directions. Anchors that are asymmetrically arranged about the midline are not mirror images of each other. Also, a single anchor structure displaced from the midline is not mirror image of another anchor structure and is an asymmetric anchor, for example anchor 236. Thus, if one were to fold the page about the midline and place the folded page together, at least one anchor structure would not overlap with another anchor structure. For example, elongate anchor 216 is located on a first side of midline 203 and no elongate anchor is located on a second side of midline 203. An alternate embodiment includes a asymmetric pair of offset anchor structures on the upper component, and each anchor structure is on an opposite side of the midline and the distance from the midline to each anchor structure is not the same; the anchor structures are asymmetric because the distance from each anchor structure to the midline is not the same and the anchor structures are not mirror images of each other about the midline.

In an alternate embodiment similar to the embodiments shown in FIGS. 2A to 2D, each component includes at least one offset anchor and the anchors are offset to the same side to avoid cutting the vertebrae along a midline. The anchors are also offset from each other to avoid cutting the vertebrae along the same plane. Laterally staggered grooves are cut in upper and lower vertebral surfaces of at least two vertically adjacent vertebral spaces, and no grove is cut on any surface on an opposite side of centerline. Thus, all of the groves are cut on the same side of the midline. At least first and second prostheses are introduced into the vertically adjacent spaces. An asymmetric anchor structure is disposed on each of the first and second prostheses and enters a grove cut into the vertebral surfaces.

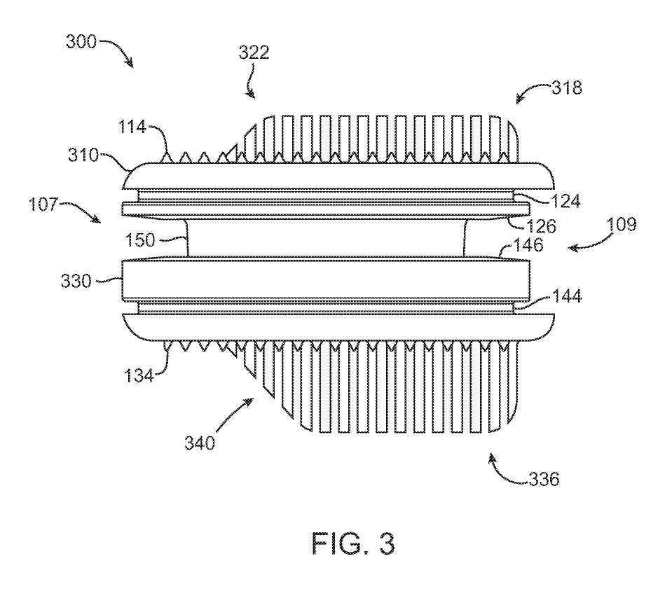

FIG. 3 shows an intervertebral prosthesis 300 with symmetric offset anchor structures comprising several pillars according to an embodiment of the present invention. Intervertebral prosthesis 300 includes an upper component 310 with an anchor structure 318 that comprises several elongate pillars disposed along a row and adapted to enter a groove formed in a patient vertebra. Anchor structure 318 is a discontinuous anchor structure. Anchor structure 318 includes several pillars of increasing height 322 to facilitate insertion of anchor structure 318 into the vertebral groove. Upper component 310 includes a second anchor structure similar and parallel to anchor structure 318. A lower component 330 comprises an anchor structure 336 formed with several elongate pillars disposed along a row and adapted to enter a groove formed in a patient vertebra. Anchor structure 336 is a discontinuous anchor structure. Intervertebral prosthesis 300 is similar to intervertebral prosthesis 100 described above and includes many similar components. Intervertebral prosthesis 300 is made from intervertebral prosthesis 100 by machining the elongate anchors 116, 118 and 136 to form several pillars from each of the elongate anchors. Pillars used as anchor structures are described in U.S. Appl. No. 60/820,770 (attorney docket no. 022031-00310US), entitled “Spinal Prosthesis with Multiple Pillar Anchors,” filed on Jul. 28, 2006, the full disclosure of which is incorporated herein by reference. Each pillar shown in intervertebral prosthesis 300 includes a gap between an adjacent pillar to permit bone growth between the pillars.

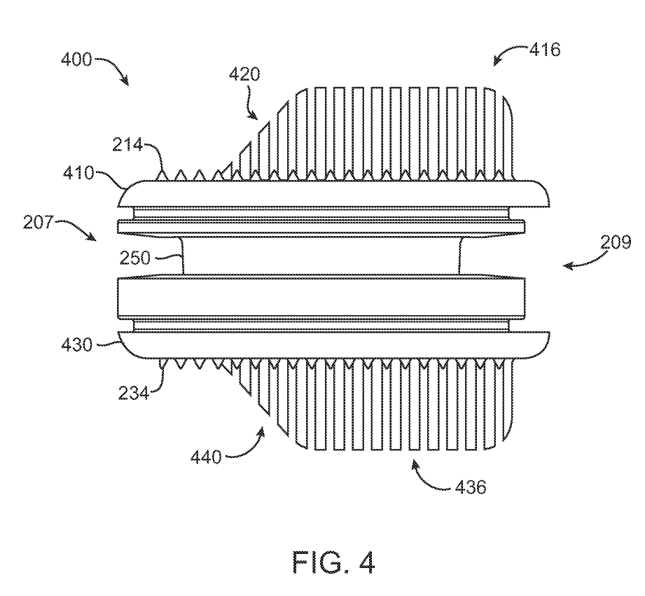

FIG. 4 shows an intervertebral prosthesis 400 with asymmetric anchor structures comprising several pillars according to an embodiment of the present invention. Intervertebral prosthesis 400 includes an upper component 410 with an anchor structure 416 that comprises several elongate pillars disposed along a row and adapted to enter a groove formed in a patient vertebra. Anchor structure 416 is a discontinuous anchor structure. Anchor structure 416 includes several pillars of increasing height 420 to facilitate insertion of anchor structure 416 into the vertebral groove. A lower component 430 comprises an anchor structure 436 formed with several elongate pillars disposed along a row and adapted to enter a groove formed in a patient vertebra. Anchor structure 436 is a discontinuous anchor structure. Intervertebral prosthesis 400 is similar to intervertebral prosthesis 200 described above and includes many similar components. Intervertebral prosthesis 400 is made from intervertebral prosthesis 200 by machining the elongate anchors 216 and 236 to form several pillars from each of the elongate anchors. Each pillar shown in intervertebral prosthesis 400 includes a gap between an adjacent pillar to permit bone growth between the pillars.

In an alternate embodiments, continuous anchor structures are combined with discontinuous anchor structures on a prosthesis. For example, the upper component shown in FIGS. 1A to 1D can be combined with the lower component shown in FIG. 3. Also, the upper component shown in FIGS. 2A to 2D can be combined with the lower component shown in FIG. 4.

While the exemplary embodiments have been described in some detail for clarity of understanding and by way of example, a variety of additional modifications, adaptations, and changes may be clear to those of skill in the art. Hence, the scope of the present invention is limited solely by the appended claims.

What is claimed is:

1. An intervertebral prosthesis comprising:
an upper component adapted to engage an upper vertebra;
a lower component adapted to engage a lower vertebra, wherein the first and second components are adapted to engage each other or an intermediate member to form an articulate joint between the vertebrae; and
an asymmetric anchor structure disposed on one of the components, the asymmetric anchor structure adapted to enter an asymmetric groove formed in the upper vertebra or the lower vertebra, wherein the prosthesis comprises a midline and the asymmetric anchor structure is disposed on a line parallel to but laterally offset from the midline.

2. The prosthesis of claim 1 wherein the asymmetric anchor structure has a height and a width, wherein the height is greater than twice the width.

3. The prosthesis of claim 1 wherein the asymmetric anchor structure comprises a continuous structure that extends in proximal and distal directions along one of the components.

4. The prosthesis of claim 1 wherein the asymmetric anchor structure comprises a fin that extends in proximal and distal directions along one of the components.

5. The prosthesis of claim 1 wherein the asymmetric anchor structure comprises a discontinuous structure that extends in proximal and distal directions along one of the components.

6. The prosthesis of claim 5 wherein the discontinuous structure comprises a plurality of anchors disposed along the parallel line.

7. An intervertebral prosthesis comprising:
an upper component adapted to engage an upper vertebra;
a lower component adapted to engage a lower vertebra, wherein the upper component and the lower component are adapted to engage each other or an intermediate member to form an articulate joint;
a first asymmetric anchor structure disposed on the upper component, the first asymmetric anchor structure adapted to enter an asymmetric groove formed in the upper vertebra, wherein the prosthesis comprises a midline and the anchor structure is disposed on a first line parallel to but laterally offset from the midline; and
a second asymmetric anchor structure disposed on the lower component, the second asymmetric anchor structure adapted to enter an asymmetric groove formed in the lower vertebra, wherein the second asymmetric anchor structure is disposed along a second line on a side of the midline opposite to that of the first asymmetric anchor structure.

8. The prosthesis of claim 7 wherein the second line is parallel to the first line.

9. The prosthesis of claim 7 wherein the first and second anchor structures are laterally offset from the midline by equal distances.

10. The prosthesis of claim 7 wherein the first and second anchor structures each has a height and a width wherein the height is greater than twice the width.

11. The prosthesis of claim 7 wherein the first and second anchor structures comprise continuous structures that extend in proximal and distal directions along the components.

12. The prosthesis of claim 7 wherein each of the first and second anchor structures comprises a fin that extends in proximal and distal directions along each component.

13. The prosthesis of claim 7 wherein the first and second anchor structures comprise discontinuous structures that extend in proximal and distal directions along the components.

14. The prosthesis of claim 13 wherein the discontinuous structures comprise a plurality of anchors disposed along each of the first and second lines.

15. An intervertebral prosthesis comprising:
a first component having at least one anchor disposed thereon, the at least one anchor adapted to enter a groove in a first vertebra;
a second component having at least one pair of anchors disposed thereon, the at least one pair of anchors adapted to enter a pair of grooves in a second vertebra, wherein the first component and the second component are adapted to engage each other or an intermediate member to form an articulate joint; and
wherein the prosthesis comprises a midline and the at least one anchor of the first component is disposed on the midline and each anchor of the at least one pair is disposed on opposite sides of the midline.

16. The prosthesis of claim 15 wherein the at least one anchor of the first component is offset from each anchor of the at least one pair of anchors.

17. The prosthesis of claim 15 wherein each anchor of the at least one pair is shorter in height than the at least one anchor disposed on the midline.

18. The prosthesis of claim 17 wherein each anchor of the at least one pair is separated from the midline by a distance, and each anchor of the at least one pair is shorter than the anchor on the midline by an amount in proportion to the distance.

19. The prosthesis of claim 18 wherein the proportional amount is from 20 to 80% of the distance.

20. The prosthesis of claim 19 wherein the proportional amount is from 30% to 70 % of the distance.

21. The prosthesis of claim 15 wherein each anchor of the at least one pair is offset from the midline by a similar distance.

22. The prosthesis of claim 15 wherein each anchor of the at least one pair has a similar height.

23. The prosthesis of claim 15 wherein the at least one anchor disposed on the midline comprises several anchors disposed on the midline and adapted to enter the groove in the first vertebra.

24. The prosthesis of claim 15 wherein the at least one pair of anchors comprises several pairs of anchors adapted to enter the pair of grooves formed in the second vertebra.

25. A method for anchoring an intervertebral prosthesis within an intervertebral space between a pair of vertebral bodies, said method comprising:
cutting a groove in a first end of at least one of the vertebral bodies, wherein the at least one vertebral body has a second opposing end with a groove which has been previously formed thereon to receive an other intervertebral prosthesis; and
introducing the prosthesis into the intervertebral space between the vertebral bodies so that at least one anchor structure enters the groove in the first end; and
wherein the groove cut in the first end is offset from the groove cut in the second end.

26. The method as in claim 25, wherein the grooves cut in the first and second ends are offset to preserve thickness of the vertebral body.

27. The method as in claim 25, wherein the grooves cut in the first and second ends are offset to avoid cutting the vertebral body along the midlines.

28. The method as in claim 25, wherein the prosthesis and the other prosthesis are disposed in adjacent intervertebral spaces separated by the at least one vertebral body with the grooves cut in the first and second ends.

29. The method as in claim 25, wherein the first groove and second groove are cut during the same surgical procedure.

30. The method as in claim 25 wherein the first groove and second groove are cut during separate surgical procedures.

31. The method as in claim 25 wherein the groove cut in the first end is cut along a first plane and the groove cut in the second end is along a second plane and wherein the first plane is offset from the second plane.

32. The method of claim 25 wherein the anchor structure is discontinuous and several anchors enter the groove in the first end as the prosthesis is introduced into the intervertebral space.

33. A method for placing intervertebral prostheses between vertically adjacent vertebrae, the method comprising:
cutting laterally staggered grooves in upper and lower vertebral surfaces of at least two vertically adjacent vertebral spaces, wherein no grove is cut on any surface on an opposite side of centerline; and
introducing at least first and second prostheses into the vertically adjacent spaces, wherein an asymmetric anchor structure on each of the first and second prostheses enter a grove cut into the vertebral surfaces.
